(19) United States
(12) Patent Application Publication
(10) Pub. No.: US 2011/0165119 A1
(43) Pub. Date: Jul. 7, 2011

(54) TELAPREVIR DOSING REGIMEN

(76) Inventors: Maria Gloria Beumont, Issy Les Moulineaux (FR); Stefan Rikard Herdinius, Mechelen (BE); Gaston Rafael Picchio, Yardley, PA (US); Ramon Polo, Yardley, PA (US); Robert S. Kauffman, Chestnut Hill, MA (US); Bambang S. Adiwijaya, Belmont, MA (US); Varun Garg, Framingham, MA (US)

(21) Appl. No.: 12/996,650

(22) PCT Filed: Jun. 10, 2009
(86) PCT No.: PCT/EP09/57222

§ 371 Date: Mar. 21, 2011

(30) Foreign Application Priority Data
Jun. 10, 2008 (EP) 08157986.4

(51) Int. Cl. A61K 38/21; A61P 31/14

(52) U.S. Cl. 424/85.4

(57) ABSTRACT

This invention relates to the use of specific dosing regimens of telaprevir in combination with peg-IFN and RBV in the treatment of HCV patients, wherein the treatment comprises (a) a lead-in phase of administering to the subject pegylated interferon and ribavirin, and (b) a treatment phase of administering to the subject a combination of telaprevir, pegylated interferon and ribavirin.

This invention relates to the use of telaprevir in combination with peg-IFN and RBV in the treatment of HCV patients. In particular, the use of specific dosing regimens of telaprevir in combination with pegylated interferon and ribavirin.

### BACKGROUND

Hepatitis C is a liver disease caused by the hepatitis C virus, which is found in the blood of people with the disease. HCV, a serious public health concern affecting 3.4 million individuals in the United States, is spread through direct contact with the blood of infected people. Though many people with HCV infection may not experience symptoms, others may have symptoms such as jaundice, abdominal pain, fatigue and fever. The burden of liver disease associated with HCV infection is increasing, and current therapies typically provide sustained benefit in less than half of patients with genotype 1 HCV, the most common strain of the virus.

The standard of care for the treatment of HCV patients consists of the combination of pegylated interferon (peg-IFN) and ribavirin (RBV). As many as 250,000 patients in the United States have received at least one course of treatment with peg-IFN and RBV but have not achieved a sustained virologic response (SVR). Patients who have failed interferon-based treatment typically have few or no available treatment options, and are at risk for rapidly progressing liver disease. The risk of liver failure, cancer or death following unsuccessful HCV treatment is 23% after 4 years, and 43% after 8 years. Re-treatment trials have shown that Peg-IFN/RBV re-treatment of subjects who failed a prior course of Peg-IFN/RBV is of limited benefit.

About 70% of acute HCV infections become persistent. Chronic HCV infection can be associated with serious liver disease such as fibrosis, cirrhosis, and hepatocellular carcinoma. HCV infection is recognized as the most common infection causing chronic liver disease and is a leading cause of death worldwide. Death from HCV infection usually occurs 20 or more years after the initial infection and it is estimated that HCV infection causes approximately 8000 to 10000 deaths each year in the US.

The ultimate goal of treatment is to eradicate the virus, thereby preventing HCV-related complications, which include, but are not limited to, decompensated liver cirrhosis and hepatocarcinoma. The likelihood of a patient achieving sustained virologic response (SVR; today defined as having undetectable plasma HCV RNA levels [<10 IU/mL] 24 weeks after the completion of treatment) has improved with the availability of long-acting pegylated interferon (Peg-IFN) plus ribavirin (RBV) treatment, with SVR rates ranging from 20 to 50% in subjects with chronic HCV genotype 1.

Despite the significant advances that have been made in the treatment of chronic HCV infection in recent years, there is an ongoing need for an effective treatment in patients who fail to achieve SVR with the current antiviral therapy. At present, the majority of patients who have received therapy for chronic hepatitis C have been treated with Peg-IFN/RBV as initial therapy or as re-treatment after a lack of response to initial therapy (defined as a <2-log decline in HCV RNA over the first 3 months of therapy or failure to achieve viral negativity or relapse following completion of treatment). It has been demonstrated that re-treating subjects with HCV genotype 1 who failed treatment with Peg-IFN/RBV results in low response rates, especially when these subjects were non-responders to prior treatment (defined as subjects who did not reach undetectable levels) as opposed to relapse subjects. There is an increasing number of patients who have failed Peg-IFN and RBV therapy, either as their initial course of treatment or as re-treatment after not achieving SVR. HCV-infected patients who have failed therapy are likely to be older and have longer disease progression than treatment-naïve patients. Patients with advanced liver fibrosis or cirrhosis (stage 3 or 4 fibrosis) are at greater risk of developing decompensated or end stage liver failure within a subsequent 5-10 year timeframe. Due to the increasing number of treatment failures, the death rate due to HCV infection is expected to increase substantially between 2009 and 2019. An estimated 232000 people in the US (8% of the total HCV-infected population) have HCV genotype 1 infection and have failed previous treatment. There is a need in the art for improved treatment of HCV patients.

Peg-IFN alfa-2b/RBV has recently been approved in Europe for patients who have failed previous treatment with IFN alfa (pegylated or non-pegylated) in combination with RBV or IFN alfa monotherapy, resulting in an SVR rate of 16 to 25%. Also the combination of Peg-IFN alfa-2a with RBV is an approved treatment. No alternative treatment with proven superiority is currently available for patients who did not achieve SVR after treatment with Peg-IFN/RBV in many regions of the world.

Telaprevir is a member of a new class of specifically targeted antiviral therapies for hepatitis C (STAT-C) and is a reversible, selective, covalent, tight, and slow-binding inhibitor of the HCV NS3-4A protease, which is essential in viral replication [WO 02/018369]. Telaprevir has structural formula (1)

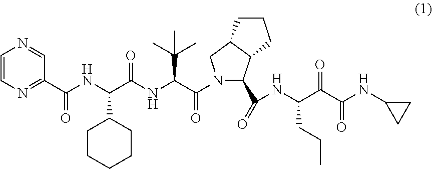

Telaprevir is a single diastereomer with the S-configuration. In vitro and in vivo, it can interconvert to its R-diastereomer to form a mixture of the 2 forms. In vitro, the R-diastereisomer is about 30 times less potent than telaprevir against the HCV NS3-4A protease. Telaprevir is orally bioavailable and may therefore be formulated in a tablet for oral administration.

Virologic breakthrough is a phenomenon defined by an increase in HCV RNA levels during treatment of more than 1 log from the lowest level achieved during treatment. Virologic breakthrough occurs on average in 5% of treatment naïve subjects that are treated with telaprevir, Peg-IFN alfa-2a and RBV. Virologic breakthroughs during telaprevir treatment are associated with telaprevir resistant variants, and the majority of them occur early in the dosing period (i.e. during the first 4 weeks). The duration of telaprevir treatment does not affect the incidence of virologic breakthrough and is not associated with an increase in the number of telaprevir-associated resistance mutations.

There is a need in the art for alternative treatments for HCV infected subject that can reduce the risk of HCV-related complications, such as hepatocellular carcinoma and decompensated liver disease, in particular for non-responders or relapsers after prior treatment. There is also a need in the art to avoid virologic breakthrough upon treatment of HCV infected subjects with telaprevir.

### SUMMARY OF THE INVENTION

The present invention concerns telaprevir administered in combination with a pegylated interferon and ribavirin, with a delayed start of telaprevir. In particular, such specific dosing regimes of telaprevir in combination with peg-IFN and RBV may generate higher SVR rates, in particular with chronic HCV genotype 1 infected subjects who may have failed prior treatment.

In one aspect, the invention relates to a combination of telaprevir with pegylated interferon and ribavirin for use in the treatment of HCV infected subjects, wherein the treatment comprises:
(a) a lead-in or initial phase of administering to the subject pegylated interferon and ribavirin, and
(b) a treatment phase of administering to the subject a combination of telaprevir, pegylated interferon and ribavirin; and optionally further comprising,
(c) a follow-on treatment phase of administering to the subject pegylated interferon and ribavirin.

In another aspect, the invention relates to a method of treating a subject infected with HCV comprising the steps of:
(a) administering to the subject pegylated interferon and ribavirin in a lead-in or initial phase, and
(b) administering to the subject a combination telaprevir, pegylated interferon and ribavirin in a treatment phase, optionally further comprising the step of:
(c) administering to the subject pegylated interferon and ribavirin in a follow-on treatment phase.

### DESCRIPTION OF THE DRAWINGS

FIG. 1 represents a schematic overview of the dosing regimens in example 1. “T” means telaprevir administration during the indicated period. “PR” means peg-IFN and ribavirin administration during the indicated period. “P” means a placebo administration for telaprevir.

### DEFINITIONS

The term “non-responders” as used herein refers to HCV patients who did not achieve an undetectable HCV RNA level after a minimum of 12 weeks of treatment with Peg-IFN and RBV.

The term “relapsers” as used herein refers to HCV patients with detectable HCV RNA during the treatment follow-up period after previous undetectable HCV RNA at end of treatment with Peg-IFN and RBV.

The term “sustained virologic response” or “SVR” as used herein refers to the situation where the patient has undetectable plasma HCV RNA levels [<10 IU/mL] 24 weeks after the completion of treatment.

For the purpose of the present invention, the terms “subject” or “infected subject” or “patient” refers to an individual infected with HCV, in need of treatment.

The term “virologic breakthrough” as used herein refers to a phenomenon defined by an increase in HCV RNA levels during treatment of more than 1 log from the lowest level achieved during treatment.

### DETAILED DESCRIPTION

The present invention relates to a combination of telaprevir with pegylated interferon and ribavirin for use in the treatment of a subject infected with HCV with delayed start of telaprevir. In particular, the combination is used for the treatment of subjects infected with HCV genotype 1. Also in particular, the presented dosage regimens are intended for the treatment of chronic HCV patients including treatment-naïve patients, non-responders or relapsers after peg-IFN/RBV therapy. Preferably, the presented dosage regimens are suitable for non-responders or relapsers. Alternatively, the presented dosage regimens are used for treatment naïve patients.

In particular, the presented dosing regimen whereby the combination of telaprevir with pegylated interferon and ribavirin is used in the treatment of HCV with delayed start of telaprevir, may prevent or reduce virologic breakthrough. The herein presented dosage regimen may reduce or prevent the occurrence of a positive selection phenomenon of telaprevir-resistant strains in the early stage of exposure to telaprevir and Peg-IFN/RBV eventually leading to virologic breakthrough.

According to an embodiment, the subject is submitted to a dosing regimen wherein during a first period, a lead-in or initial phase, peg-IFN and ribavirin are administered, followed by a second period, a telaprevir treatment phase, wherein telaprevir is administered in combination with peg-IFN and ribavirin. According to a particular embodiment, said telaprevir treatment phase is further followed by a third period, a follow-on treatment phase, wherein peg-IFN and ribavirin are administered.

In particular, there may be no time lag between the lead-in or initial phase and the telaprevir treatment phase, or there may be no time lag between the telaprevir treatment phase and the follow-on phase. More in particular, there is no time lag between the lead-in phase and the telaprevir treatment phase and between the telaprevir treatment phase and the follow-on phase. No time lag between treatment phases means that the respective treatment phases follow each other directly, that there is no treatment interval. For example, when a first treatment phase ends, the next treatment phase starts directly thereafter, e.g. the next day.

The presented telaprevir dosage regimen with lead-in phase may also be referred to as delayed start telaprevir treatment.

Within the same embodiments, said first period may take up to six weeks, in particular said first period may take up to five weeks. Also in particular, said first period may take at least two weeks, in particular at least three weeks. More in particular, said first period takes about four weeks. Also within the same embodiments, the second treatment period may take at least eight weeks, preferably at least ten weeks. Also in particular, said second treatment period may take at most 16 weeks, preferably no more than 14 weeks. More in particular, said second treatment period takes about 12 weeks. Also within the same embodiments, said second treatment period may take at least 26 weeks, in particular at least 30 weeks. Also, said third treatment period may take at most 36 weeks, in particular said third treatment period may take at most 34 weeks. More in particular, said third treatment period takes about 32 weeks.

It should be understood that lower and higher limits for the same purpose might be combined to provide preferred ranges.

For the dosage regimens according to the embodiments herein, on the indicated days for administration of telaprevir, telaprevir may be administered twice, three times or four times a day. Telaprevir may be administered in an amount of about 300 mg to about 1500 mg, in an amount of about 300 mg to about 1250 mg, in an amount of about 450 mg, in an amount of about 1250 mg, or in an amount of about 750 mg. Telaprevir may also be administered in an amount of about 1125 mg. Telaprevir may typically be administered in a dose of 750 mg three times a day, specifically in a dose of 750 mg every 8 hours. Alternatively, telaprevir may typically be administered in a dose of 1125 mg twice a day, specifically, in a dose of 1125 mg every 12 hours.

### Example 1

A randomized, double-blind, placebo-controlled study is conducted with telaprevir in subjects with chronic HCV genotype 1 infection who failed prior treatment with Peg-IFN (Peg-IFN alfa-2a or Peg-IFN alfa-2b) plus RBV. The trial is designed to compare the efficacy, safety, and tolerability of 2 regimens of telaprevir (with and without delayed start) combined with Peg-IFN alfa-2a and RBV versus standard treatment (Peg-IFN alfa-2a and RBV).

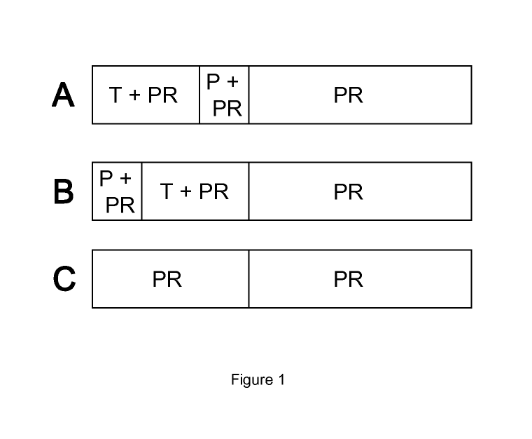

The trial consists of a screening period of approximately 4 weeks, a 48-week treatment period, and a 24-week follow-up period. A schematic overview of the design of the experiment is presented in FIG. 1.

Subjects taken up in the study meet either one of the following criteria:
(1) subject had an undetectable HCV RNA level at the end of a prior course of Peg-IFN/RBV therapy but did not achieve SVR (viral relapsers), or
(2) subject never had an undetectable HCV RNA level during or at the end of a prior course of Peg-IFN/RBV therapy (non-responders).

Subjects will be randomized to one of 3 treatment groups: 2 telaprevir regimens (Treatment group A, without delayed start of telaprevir, and Treatment group B, with delayed start of telaprevir) and one control group (Treatment group C). All 3 treatment groups have a planned treatment duration of 48 weeks. In both telaprevir regimens (A and B), subjects receive 12 weeks of 750 mg telaprevir every 8 hours (referred hereinafter as “q8h”) in combination with 48 weeks of Peg-IFN alfa-2a (Pegasys) and RBV (Copegus) at standard doses. In Treatment group B, treatment with telaprevir has a delayed start: telaprevir treatment starts 4 weeks after the start of Peg-IFN alfa-2a and RBV treatment. In the control group (C) subjects receive Peg-IFN alfa-2a and RBV at standard doses for 48 weeks. Standard doses for Peg-IFN alfa-2a is 180 μg/week. Standard doses for RBV is 1000 mg daily for subjects weighing less than 75 kg, and 1200 mg daily for subjects weighing 75 kg or more. RBV is typically administered orally in a twice daily regimen.

A detailed overview of the treatments in the 3 treatment groups and the planned number of subjects is given below:
Treatment group A (260 subjects, i.e., 140 relapsers and 120 non-responders):
   telaprevir in combination with Peg-IFN alfa-2a and RBV for 12 weeks; followed by
   placebo in combination with Peg-IFN alfa-2a and RBV for 4 weeks; followed by
   Peg-IFN alfa-2a and RBV for 32 weeks.
Treatment group B (260 subjects, i.e., 140 relapsers and 120 non-responders):
   placebo in combination with Peg-IFN alfa-2a and RBV for 4 weeks; followed by
   telaprevir in combination with Peg-IFN alfa-2a and RBV for 12 weeks; followed by
   Peg-IFN alfa-2a and RBV for 32 weeks.
Treatment group C (control group; 130 subjects, i.e., 70 relapsers and 60 non-responders):
   placebo in combination with Peg-IFN alfa-2a and RBV for 16 weeks; followed by
   Peg-IFN alfa-2a and RBV for 32 weeks.

Randomization is stratified to optimize balance between treatment groups with regard to prior virologic response. Approximately 350 relapsers and approximately 300 non-responders are included.

Furthermore, specifically for the stratum of prior non-responders, an additional stratification is done based on type of prior non-response. Enrollment of the subjects is limited such that within the subgroup of non-responders neither of the following strata represents more than 55%:
subjects who had <2 log drop in HCV RNA at Week 12 of previous therapy (null-responder);
subjects who had 2 log drop in HCV RNA at Week 12 of previous therapy, but who never achieved undetectable HCV RNA levels while on treatment (partial responder).

Telaprevir is formulated as a caplet-shaped tablet for oral administration, containing 375 mg of telaprevir in combination with pharmaceutically acceptable carriers. When applicable, Telaprevir is administered in a dose of 750 mg every 8 hours.

Telaprevir matching placebo tablet for oral administration consists of a mixture of lactose anhydrous, microcrystalline cellulose (Avicel PH102), magnesium stearate, and FD&C yellow dye #5/tartrazine.

Peg-IFN alfa-2a is formulated as a 180-μg solution for subcutaneous injection in a pre-filled syringe also containing pharmaceutical carriers. When applicable, peg-IFN alfa-2a is administered in a dose of 180 μg once a week. RBV is formulated as a film-coated tablet for oral administration containing 200 mg of RBV. When applicable, RBV is administered in a dose of 1000 mg (for subjects weighing less than 75 kg) and 1200 mg (for subjects weighing 75 kg or more) in two gifts per day.

1. Combination of telaprevir with pegylated interferon and ribavirin for treatment of HCV infected subjects, wherein the treatment comprises:
(a) a lead-in phase of administering to the subject pegylated interferon and ribavirin, and
(b) a treatment phase of administering to the subject a combination of telaprevir, pegylated interferon and ribavirin,
wherein there is no time lag between the lead-in phase and the treatment phase.

2. Combination according to claim 1 wherein the treatment further comprises
(c) a follow-on treatment phase of administering to the subject pegylated interferon and ribavirin.

3. Combination according to claim 2 wherein there is no time lag between the treatment phase and follow-on treatment phase.

4. Combination according to claim 1 wherein the HCV infected subjects are infected with HCV genotype 1.

5. Combination according to claim 1 wherein the HCV infected subject is a relapser or a non-responder.

6. Combination according to claim 1 wherein the HCV infected subject is treatment naïve.

7. Combination according to claim 1, wherein the lead-in phase takes between two and 6 weeks.

8. Combination according to claim 7, wherein the lead-in phase takes 4 weeks.

9. Combination according to claim 1, wherein the treatment phase takes between eight and sixteen weeks.

10. Combination according to claim 9, wherein the treatment phase takes 12 weeks.

11. Combination according to claim 2, wherein the follow-on treatment phase takes between 26 and 36 weeks.

12. Combination according to claim 11, wherein the follow-on treatment phase takes 32 weeks.

13. Combination according to claim 1, wherein during the treatment phase, telaprevir is administered in an amount of 750 mg every 8 hours.
